(19) United States
(12) Patent Application Publication
(10) Pub. No.: US 2023/0061309 A1
(43) Pub. Date: Mar. 2, 2023

(54) Artificial intelligence application that identifies micro-organisms, matter and contaminates from water sources

(71) Applicant: Keith Louis DeSanto, St Petersburg, FL (US)

(72) Inventor: Keith Louis DeSanto, St Petersburg, FL (US)

(21) Appl. No.: 17/411,379

(22) Filed: Aug. 25, 2021

(51) Int. Cl. C12Q 1/04; C12M 1/26; C12M 1/00; C12M 1/12; B01L 3/00; G06N 20/00

(52) U.S. Cl. CPC C12Q 1/04; C12M 33/00; C12M 39/00; C12M 23/04; B01L 3/508; G06N 20/00

(57) ABSTRACT

This new artificial intelligence application identifies micro-organisms, matter and contaminates from the bottom of lakes, rivers, harbors, streams and any other large bodies of water utilizing conveyor belts with and without embedded microscope slides, watertight containers, high definition lenses, drones, robots, artificial intelligence and machine learning algorithms. The AI application can be used below and above the surface of water. The AI command center utilizes a gorilla glass panel to manipulate by finger or hand to be viewed clearly by the human eye the matter, microbes and contaminates on the glass before and after they have been analyzed by the algorithms and servers.

This new artificial intelligence application identifies micro-organisms, matter and contaminates from the bottom of lakes, rivers, harbors, streams and any other large bodies of water including seabeds. The application utilizes a combination of dredging equipment, claws, pumps, hoses, buckets, containers and mechanical arms to transport debris, mud, sludge, water, matter, contaminates, micro-organisms or combinations of hereafter referred to “dredged material or watery slush”. Depending on the specific application, the microbes and matter are pumped, dropped, placed, sprayed or flowed onto a conveyor belt embedded with stacked microscope slides and or microscope plates under the water or above the surface. The conveyor belts maintain electron microscopes, light microscopes, x-ray machines and high definition lenses for picture and video taking of dredged material or watery slush on the slides and plates. The lenses of the microscopes may utilize the base of the entire microscope apparatus, parts of microscopes such as the lenses and the condensers or just the high definition lenses may be used on the conveyor belts. All prior listed lenses can be over, under, through or on both sides of the conveyor belt. The data from the lenses is then transferred to servers for processing through the micro-organism and matter facial recognition algorithms. The entire application is automated and managed by artificial intelligence and machine learning algorithms. A command center can also be used utilizing a gorilla glass panel to manipulate by finger or hand to view the matter and microbes transported to the surface by the human eye on the glass after they have been analyzed by the servers and algorithms.

The application of identifying dredged material or watery slush is just that, an application to identify microbes, matter and contaminates and after accurate identification, the application can also pinpoint what type of microbe, matter or contamination is present and propose solutions to eliminate them. The machine learning algorithms learns from scans, dredging, dredged material and behaviors below the surface of the water and utilizes prediction algorithms to forecast some events.

The application utilizes lights, fans, vibrations, air and liquid pressure and tilting of slides, plates and conveyor belts to position micro-organisms and matter from water floors on conveyor belts with embedded microscope slides and plates. The slides and plates are viewed by electron microscopes, light microscopes, x-ray machines and high definition lenses placed above, below and on both sides of the conveyor belts.

The application can work both under water and above water. The underwater application utilizes a closed system sitting on a floor of a body of water (or 1 to 5 feet suspended by a platform above the floor-depending on the level of debris in the water) and the above the surface of the water application on a ship or on land. The platform can be affixed to the floor of the body of water by numerous ways including but not limited to spikes driven far into the water floor, tables with weights on the bottom of the legs or tables made of concrete. The platform when on land can be affixed to tables and set in concrete.

The underwater application can have many separate containers stacked on top of each other. The first container (sitting on the top of the others) allows the dredged material or watery slush to pass through the sides of the container with screens (the screens block out large matter) by a pump outside one side of the container or on the top of the container. The matter can also be pumped into and out the top container by an internal pump inside or on the inside of the top of the container. After each run, each container can be cleaned by semi filtered water that is pulled in from 1 to 20 feet above the container. The artificial intelligence platform determines if the pumps should reverse the flow of water for cleaning purposes.

After the dredged material or watery slush is pumped into the top container, the container closes and the floor of the top container opens into the second container that houses a conveyor belt with some or all of the hardware listed above. The dredged material or watery slush is manipulated as described below. After data is collected and transferred from the middle (second container), the third container where the dredged material or watery slush is dropped again through the floor and the process is then restarted. There can be many stacked containers, the more containers, the more accurate the data becomes.

If any of the containers becomes clogged, purified water or water from 1 to 20 feet above the top container is pumped into the container to free the dredged material or watery slush or foreign material. If a clogged container cannot be cleaned by this manner, a robot stands by to assist with a mechanical arm that is housed underneath the table. The AI platform manages this process by water flow meters inside the containers.

The artificial Intelligence and machine learning algorithms “AI and ML” manage the entire system where the platform would determine if air, vibration, magnetics, titling or more of a sample or cleaning is needed for accurate identification purposes.

The above water application works in 2 separate ways, mobile and stationary with the stationary application having greater accuracy.

The Mobile application. After the dredged material or watery slush has been pumped or transferred on to the surface on a ship or land, robots and drones imbedded with high definition lenses with and without condensers take samples by the splashing method. The lenses are covered with shutters and the drone and or robot utilizes a mechanical arm to splash the slide or plate with the dredged material or watery slush from the bottom of the drone or robot. After splashing, the slide is placed below the lens and the data is transferred wirelessly to a desktop or laptop. The slide or plate is then dipped in a pool of water for cleaning by the mechanical arm for reuse.

Another mobile drone and robot transport and identification method is the following. Using plates and slides that sit directly under the drone and or robot where they instantly press against the dredged material or watery slush. A slide or plate is pressed to the dredged material or watery slush by the drone or robot for a short time as determined by the AI/ML platform. This method is called the pressing method or also called the plate and slide pressing method. The slide or plate is pressed to dredged material or watery slush many times. The drones and or robots transport that slide or plate over the conveyor belt. The slide is then dropped onto a conveyor belt without imbedded slides and plates for processing. The drones and robots use either a mechanical arm or magnetics to pick up or drop off the plate to the conveyor belt.

The data from the dredged material or watery slush is then transferred to laptops desktops and then to servers for analysis, evaluation and the microbe and matter facial recognition algorithms identifies from a data base of pictures and videos by comparison. The database on servers maintains trillions of pictures from prior applications in specific bodies of water.

Another mobile application is to specifically transport the slides or plates by drones and robots to the top of the embedded slides and plates on a conveyor belt. The slides when placed on top of the embedded slide or plate on the conveyor belt locks microbes between the slides. This application is used for strictly identifying micro-organisms in water. This application is referred to as “Locking Water Slides” the “LWS” app utilizes magnetics to place and retrieve slides and plates from and to the conveyor belt.

Both drones and robots are equipped with small tanks for liquid, biosurfactants and air to clean the lenses if needed. Some drones and robots are equipped with shutters that cover the lenses and cameras that can assist in finding large and small pools of dredged material or watery slush when not operating.

The mobile application also utilizes the conveyor belt application and mechanical arms on drones and robots. The drones and robots deposit or drop dredged material or watery slush samples from their mechanical arms on the edge of the conveyor belts where the splash will cause some matter to come into contact with the conveyor belt and some of the dredged material or watery slush make their way on top of the embedded slides or plates on the conveyor belt equipped with high definition lenses to take video and pictures of the matter and microbes and contaminates from the dredged material or watery slush. The data is again transferred to servers and algorithms.

Both drones and robots utilize mechanical arms to place the dredged material or watery slush onto the conveyor belt where the application is time consuming.

The stationary application uses many conveyor belts equipped with electron microscopes, light microscopes, x-ray machines and high definition lenses. The lenses may use the original base the light and electron microscope came with being on both sides of the conveyor belt (lenses on top of the conveyor belt and the condenser below the belt), with the base removed and with just the lens and condenser on both sides of the conveyor belt (top and bottom or vice versa). The stationary application utilizes our flooding and grate procedure. The conveyor belt is affixed to the floor of a ship or affixed to a metal, concrete or wood box or table on land. A grate or screen is used in the following manner. Matter is pumped from the floor of the body of water where the sludge is a ratio of water and debris to create a watery sludge. Watery sludge is defined as mud, gravel, debris, micro-organisms, contaminates and matter dredged from the bottom of a water bed. The watery sludge flows evenly on a tray slightly titled to have some of the watery sludge flow onto a moving conveyor belt. The conveyor belt is also embedded with slides and plates where high definition lenses with and without condensers, electron microscopes, light microscopes or a combination of all take pictures and video of micro-organisms and matter that are then transferred to a server or servers with specific algorithms. The application is accurate due to the millions and millions of pictures and video transferred to servers that use microbe and matter recognition algorithms and databases to identify such. The belt continues one loop with between 10 and 10,000 pictures and video depending on the size of the conveyor belt(s) and the number of lenses involved with the conveyor belt. If the AI platform decides that another rotation is needed, the conveyor belt will make another loop and continue until accurate pictures and or video is obtained. At times, hundreds or thousands of pictures and or video may be needed for accurate identification of the dredged material or watery slush. If more data is required by the AI platform, more watery sludge may be added to the tray and the process will be restarted. The fluids are continuously recycled until the same material is seen 5 times each and then the AI platform moves onto a new tray of watery sludge. This process may continue for quite some time or until accurate identification of micro-organisms, matter and contaminates are obtained.

The conveyor belt is able to tip itself slightly to remove the majority of the water mud sediment therefore dripping off of the side. Some conveyor belts allow for plates and slides to slightly tip to remove excess dredged material or watery slush for greater accuracy. The AI platform may also determine if more slushy water, tap water or purified water is needed to be applied to the conveyor belt for flowing purposes. Some conveyor belts have cameras affixed to them to determine flow, clogging or buildup of dredged material or watery slush needs to removed, watered down or emulsified with biosurfactants. The AI platform utilizes a manipulation platform for high accuracy of data where the application can also use the exascale method.

The debris is allowed by water flow to remain on the slide and or plate embedded in a conveyor belt with electron microscopes, light microscopes and or high definition lenses with and without condensers to take pictures and or video of each slide or plate. Those pictures and video can be transferred in pieces of data (if they are in large files) or in full large file transfer to laptops, cell phones, desktops or tablets who will then transfer that data to a server or many servers on land (connected or stand-alone) to evaluate the obtained data using matter and micro-organism and debris “facial” recognition technology database algorithms.

The conveyor belt managed by the AI platform will determine if vibration of the conveyor belt, magnetic pulses and or charges are required, blasts of air and or liquids including rhamnolipid to position matter on a slide or plate for identification directly below a lens.

The dredged material or watery slush is obtained by a dredging machine. The application utilizes dredging machines of various sizes to transport water, soil, sediments, pieces of and or debris (material) and contaminates “dredged material or watery slush” from below the surface of water of lakes, rivers, harbors, streams and any other water bodies. Water, soil, rock, shells or anything that can be pumped to the surface from the floor of a body of water for identification of micro-organisms, matter and contaminates will be analyzed by the application. The dredged material or watery slush can also be obtained by shocking or creating a wave to obtain the sample.

After the dredged material or watery slush is transported by pipe, machine, current or by hand, the dredged material or watery slush sample is allowed to flow over the conveyor belt (then stopped and restarted again) where the amount of starts, stops and restarts enable the system to collect data via pictures and video to send to the servers for evaluation by artificial intelligence and machine learning algorithms.

Cleaning of the application for reuse or transport. Replacing of lenses, slides and plates.

The system cleans itself or replaces the lenses, plates and slides.

The self-cleaning application operates in two ways: blasts of clean water onto the plates or slides. Or air is blasted with and without a liquid such as rhamnolipid.

On more expensive applications, the slides and plates can be stacked when embedded on the conveyor belts therefore allowing the plates and slides to be removed or dropped off when they become scratched. The dropped plates and slides are gathered by drones and or robots and refurbished for later use.

Drone and robots as managed by the AI platform utilize mechanical arms (mechanical cloth arms) that are used on the plates and or slides to clean for reuse. Some slides can be diamond coated or made exclusively of diamond to avoid scratches. The stand-alone lenses or electron microspore lenses or light microscope lenses when required utilize a water and air tight shutter the cover the lenses when the conveyor belt is being flooded with debris. The shutter is usually utilized in salt water applications but also can be used in brackish and fresh water applications. The drones and or robots will replace lenses when the AI application determines so.

The application analyses and evaluates copious amounts of data enabling accurate identification of micro-organisms and matter from material obtained from bodies of water.

The machine learning algorithms not only learns about specific bodies of water but also forecasts what may transpire in the future. An example of this is a storm of high winds and flooding will cause other microbes and matter to enter the body of water and the platform may warn against drinking the water, swimming in it or for other use.

A central command center for all worldwide applications.

The artificial intelligence and machine learning application also offers a cockpit command center for large applications in large bodies of water such as Lake Erie.

The cockpit utilizes gestural control of files, text, pictures and video on a clear screen with dimensions of 3 feet by 3 feet and larger.

Using see through “Gorilla” glass to project and organize microbe and matter pictures and associated data, the transferred data after being analyzed by servers around the world, can be managed by moving files, text, video and pictures around a giant glass screen by waving a hand, touching the screen with an object or a finger, a human body part or by voice.

A user interface made of glass (silicone or Gorilla Glass) that can be maneuvered by a touch of a hand or finger or managed by voice (artificial intelligence) to project, manage or organize pictures and video of micro-organisms and matter allowing the magnification of such pictures and video to be seen by the human eye before and after being analyzed by algorithms on servers. Moving pictures and video on a giant screen in front of a person will make the management easier for operator and client.

1. An application that transports dredged material and watery slush from the bottom of lakes, rivers, harbors, streams or any other large body of water to a platform of conveyor belts embedded with microscope slides and plates, light microscopes, electron microscopes, x-ray machines or high definition lenses with and without condensers where the entire platform is managed by artificial intelligence and machine learning algorithms for identification purposes.

2. An application that utilizes stacked containers that are equipped with conveyor belts embedded with microscope slides and microscope plates, high definition lenses and condensers, lights, internal and external pumps, robots with mechanical arms and transmission equipment on the floor of a body of water that is tethered to a ship on the surface of the water or is tethered to a stationary platform on land to identify micro-organisms, matter and contaminates using artificial intelligence and machine learning algorithms.

3. An application whereas drones and robots above the surface of a body of water or on land utilize a direct wireless charging system to transport, process and identify micro-organisms, matter and contaminates using conveyor belts from dredged material from a floor of a body of water that are managed by an artificial intelligence platform with machine learning algorithms.

4. An application in claims 1 and 2 that utilizes artificial intelligence and machine learning algorithms to manage fans, lights, cameras, vibrators that cause vibrations, air and liquid pressure nozzles and tilting on the conveyor belt to position micro-organisms, matter and contaminates for identification.

5. An application in claim 3 where drones and robots deposit or drop dredged material or watery slush to the edge or top of a conveyor belt embedded with microscope slides and microscope plates to identify micro-organisms, matter and contaminates.

6. An application in claim 1 where dredged material or watery slush is pumped from, retrieved from or transported from below the surface of a body of water to above the surface of water or on land to a tray that is slightly titled to have some of the dredged material or watery slush flow onto a moving conveyor belt.

7. An application in claim 6 where a drone and or a robot presses a plate or slide to the dredged material or watery slush for identification.

8. An application in claims 1 through 3 where artificial intelligence and machine learning algorithms will determine if vibration, magnetic pulses, blasts of air and or liquids are needed to position matter on a slide or plate of a conveyor belt for identification directly below a high definition lens.

9. An application in claims 1 through 3 where algorithms that recognize micro-organisms, matter and contaminate from lakes, rivers, harbors, streams or any other large bodies of water utilizes that information and data to forecast events that may take place below the surface of water.

10. An application in claims 1 through 9 where the cause and type of water contamination is identified.

11. An application in claim 2 where algorithms for identifying micro-organism, matter and contamination are used from past prior facial recognition data stored on servers for matching with the new data of pictures and or video.

12. An application in claim 1 allowing for plates and slides to placed, removed or dropped from the air on a conveyor belt by drones and robots with or without mechanical arms.

13. An application in claims 1 through 3 where a conveyor belt or the embedded plates and slides are able to separately tip themselves slightly to remove the majority of the dredged material and slushy water and then return to being level for micro-organism, matter and contaminate identification.

14. An application in claim 2 where the sides of stacked containers maintain grates or screens where the sides open and close, the bottom of the container also opens and closes to allow for dredged material or watery slush to flow through the sides and bottom of the containers to containers that hold conveyor belts, high definition lenses, lights, pumps (internal and external) where robots with mechanical arms await below the container to aid in case of a clogg.

15. An application in claim 14 the containers are set on heavy tables that hold robots with mechanical arms where a pipe or hose pulls in and forces water out of the container water that is 1 to 20 feet above the container.

16. An application in claims 1 through 14 where the application is managed by an artificial intelligence platform which include machine learning algorithms to determine the surrounding characteristics of the area utilizing past data, present data and predicting future data.

17. An application in 1 through 15 where the lenses are covered by an airtight and water tight shutter that covers the lenses where artificial intelligence with machine learning algorithms manage the shutters.

18. An application in claim 17 where drone and robots as managed by the AI platform that utilize mechanical arms with cloth that are used on the diamond plated plates and or slides to clean for reuse.

19. An application in claims 1 through 18 and claim 20 where a command center using see through “Gorilla” glass is used to project and organize microbe and matter pictures and their associated data by moving files, text, video and pictures around a giant glass screen by waving a hand, touching the screen with an object or a finger, a human body part or having the images moved by voice.

20. An application in claims 1 through 19 where the sizes of the components are either nano-technology sized, normal “contemporary” sized or a combination of both.
